(19) United States
(12) Patent Application Publication
(10) Pub. No.: US 2018/0140645 A1
(43) Pub. Date: May 24, 2018

(54) PROBIOTIC OR PREBIOTIC, METHOD FOR PRODUCING SAME, MICROBIAL PREPARATION, HEALTH FOOD, AND MEDICINE

(71) Applicants: JAPAN ECO-SCIENCE CO., LTD., Chiba-shi, Chiba (JP); KEIYO PLANT ENGINEERING CO., LTD, Ichikawa-shi Chiba (JP); MIROKU CO., KTD., Oita (JP); NATIONAL UNIVERSITY CORPORATION CHIBA UNIVERSITY, Chiba-shi, Chiba (JP)

(72) Inventors: Hirokuni MIYAMOTO, Chiba-shi, Chiba (JP); Hiroaki KODAMA, Chiba-shi, Chiba (JP); Hiroshi OHNO, Yokohama-shi Kanagawa (JP); Shinji FUKUDA, Yokohama-shi Kanagawa (JP); Masahira HATTORI, Kashiwa-shi Chiba (JP); Kenshiro OSHIMA, Kashiwa-shi Chiba (JP); Wataru SUDA, Kashiwa-shi Chiba (JP); Toshiyuki ITO, Ichikawa-shi Chiba (JP); Hisashi MIYAMOTO, Oita (JP)

(21) Appl. No.: 15/578,281

(22) PCT Filed: Mar. 4, 2016
(86) PCT No.: PCT/JP2016/056806

§ 371 Date: Nov. 30, 2017

(30) Foreign Application Priority Data
May 30, 2015 (JP) 2015-121531

(51) Int. Cl. A61K 35/742; A61P 1/00; A61P 31/04; A23L 31/15

(52) U.S. Cl. CPC A61K 35/742; A23L 31/15; A61P 31/04; A61P 1/00

(57) ABSTRACT

The diatheses of animals vary depending on genetic backgrounds of the animals, and the microbial structure of an enterobacterial flora inherent in a host and the behavior of the concentration of a biological molecule sometimes vary depending on the diathesis of the host. In this case, it is needed to administer a proper probiotic.

Provided is a microbial preparation for controlling the proportion of the population of bacteria belonging to the division Bacteroidetes, Firmicutes or Proteobacteria or the proportion of the population of bacteria belonging to the genus Clostridium, Lactobacillus, Bifidobacterium or Bacteroidetes in the enterobacterial florae in an animal body, and for controlling the concentration of a functional molecule contained in a living body, said microbial preparation containing a microorganism P01931 (International Accession No. BP-1931), MK-01A (International Accession No. BP-02066) or MK-03A (International Accession No. BP-02067) or a component of the microorganism.

### TECHNICAL FIELD

In the present invention, attention is focused on the matter that a thermophilic bacterium produced using a thermophilic bacterium exhibiting different functions depending on the diatheses of animal bodies and a mixed solution containing the thermophilic bacterium have a function to cause different physiological reactions depending on genetic backgrounds of the animal bodies and diatheses of an enterobacterial flora. Thus, the present invention relates to a preparation utilizing the function and a method for producing the preparation.

### BACKGROUND ART

As preparations for controlling the intestinal function in an animal body in good condition, probiotics and prebiotics are known. As the techniques for the preparations, techniques which utilize microorganisms inhabitable predominantly at ambient temperature, such as lactic acid bacteria, yeast fungi and grass bacillus, have been widely used (Patent Literature 1, Patent Literature 2). For example, Patent Literature 1 discloses a method for producing a non-fermentative lactic acid bacterium using a bifidobacterium and Lactobacillus acidophilus; and Patent Literature 2 discloses a preparation for controlling the secretion of adiponectin using a culture supernatant of a lactic acid bacterium Lactobacillus gasseri SBT2055 (FERM BP-10953). Patent Literature 3 discloses an Anoectochilus spp. polysaccharide extract and a pharmaceutical composition both for stimulating the growth of a bacterium belonging to the genus Bifidobacterium, stimulating the release of a granulocyte colony-stimulating factor, promoting the differentiation of T helper cell type I and/or suppressing the differentiation of T helper cell type II, and methods respectively for preparing the extract and the pharmaceutical composition, wherein the techniques relating to the Anoectochilus spp. polysaccharide extract and the pharmaceutical composition both for stimulating the growth of a bacterium belonging to the genus Bifidobacterium, stimulating the release of a granulocyte colony-stimulating factor, promoting the differentiation of T helper cell type I and/or suppressing the differentiation of T helper cell type II are introduced.

As Non Patent Literatures, a worldwide study in which, among bacteria species belonging to the genus Clostridium, bacteria species and bacteria both involving in the control of immune system cells are identified is known (Non Patent Literature 1). Also known are segmented filamentous bacteria (SFB) which are specific bacteria capable of regulating immune systems (Non Patent Literature 2) and an extremely important study for searching for an effective gene capable of protecting against 0157 in a bifidobacterium (Non Patent Literature 3).

In these techniques, however, the control of enterobacterial florae depending on the types of the enterobacterial florae with genetic backgrounds of hosts taken into consideration is not referred. In recent years, study data which demonstrate worldwide that both of feeds and sexual difference affect the constitution of an intestinal microbial flora have been reported (Non Patent Literature 4), and it has been shown that the diversity of an enterobacterial flora is important for the measure against metabolome in human bodies (Non Patent Literature 5). Therefore, it is considered that it will be needed in the future to design a combination of proper probiotics on the basis of the genetic backgrounds of hosts.

On the other hand, the present inventors have succeeded in the establishment of techniques for probiotics having influence on animal living bodies by using thermophilic bacteria which are one type of extremophiles capable of hardly proliferating in an ambient temperature range (Patent Literatures 4 and 5, and Non Patent Literature 5).

### CITATION LIST

### Patent Literatures

Patent Literature 1: Japanese Patent No. 4898859
Patent Literature 2: Japanese Patent No. 5225652
Patent Literature 3: Japanese Patent No. 5395733
Patent Literature 4: Japanese Patent No. 5578375
Patent Literature 5: Japanese Patent No. 5041228

### Non Patent Literatures

Non Patent Literature 1: Atarashi K, Tanoue T, Oshima K, Suda W, Nagano Y, Nishikawa H, Fukuda S, Saito T, Narushima S, Hase K. Kim S, Fritz J V, Wilmes P, Ueha S, Matsushima K, Ohno H, Olle B, Sakaguchi S, Taniguchi T, Morita H, Hattori M, Honda K*. “Treg induction by a rationally selected mixture of Clos tridia strains from the human microbiota.” Nature 500:232-236. (2013)
Non Patent Literature 2: Ivanov II, Atarashi K, Manel N, Brodie E L, Shima T, Karaoz U, Wei D, Goldfarb K C. Santee C A, Lynch S V, Tanoue T, Imaoka A, Itoh K, Takeda K, Umesaki Y, Honda K*. Littman D R*. “Induction of intestinal Th17 cells by segmented filamentous bacteria.” Cell. 139:485-98. (2009)
Non Patent Literature 3: Fukuda S, Toh H, Hase K, Oshima K. Nakanishi Y, Yoshimura K, Tobe T, Clarke J M, Topping D L, Suzuki T, Taylor T D, Itoh K, Kikuchi J. Morita H, Hattori M, Ohno H. Bifidobacteria can protect from enteropathogenic infection through production of acetate. Nature. 2011 Jan. 27:469(7331):543-7.
Non Patent Literature 4: Bolnick D I, Snowberg L K. Hirsch P E, Lauber C L, Org E, Parks B, Lusis A J, Knight R, Caporaso J G, Svanb&auml; ck R Individual diet has sex-dependent effects on vertebrate gut microbiota. Nat Commun. 2014 Jul. 29:5:4500 doi: 10.1038/ncomms5500.
Non Patent Literature 5: Le Chatelier E, Nielsen T, Qin J, Prifti E, Hildebrand F. Falony G. Almeida M, Arumugam M, Batto J M, Kennedy S, Leonard P. Li J. Burgdorf K. Grarup N, Jorgensen T, Brandslund 1, Nielsen H B, Juncker A S, Bertalan M. Levenez F, Pons N, Rasmussen S, Sunagawa S, Tap J, Tims S, Zoetendal E G, Brunak S. Clement K. Dore J, Kleerebezem M, Kristiansen K, Renault P, Sicheritz-Ponten T, de Vos W M, Zucker J D, Raes J. Hansen T; MetaHIT consortium, Bork P, Wang J, Ehrlich S D, Pedersen O. Richness of human gut microbiome correlates with metabolic markers. Nature 500:541-549. (2013)
Non Patent Literature 6: Miyamoto H. Seta M, Horiuchi S, Iwasawa Y, Naito T, Nishida A. Miyamoto H. Matsushita T, Itoh K. Kodama H (2013) Potential probiotic the rmophiles isolated from mice after compost ingestion. Journal of Applied Microbiology. 114(4): 1147-1157

### SUMMARY OF THE INVENTION

### Technical Problems

In the conventional techniques, the idea of administering different probiotics to different animals depending on diatheses of the animals has not been established yet. However, it is known that bacterial florae that form intestinal environments as well as genetic backgrounds are greatly vary depending on the species of animals. Therefore, probiotics or prebiotics which can be utilized with the above-mentioned situations taken into consideration may be demanded.

The diatheses of animals vary depending on genetic backgrounds of the animals, and the microbial structure of an enterobacterial flora inherent in a host and the behavior of the concentration of a biological molecule sometimes vary depending on the diathesis of the host. An antibiotic is sometimes used as a control for an enterobacterial flora. In this case, however, it is concerned that the diversity of an enterobacterial florae may be lost and an adverse effect on intestinal environments may be developed. In these cases, it is needed to administer a proper probiotic.

The purpose of the present invention is to provide a probiotic or prebiotic that can solve the above-mentioned problems.

### Solution to Problems

A thermophilic bacterium probiotic which can act depending on the properties of animals of strains having susceptibility to fatness and animals of strains having insusceptibility to fatness is used. By using this probiotic, the population of enterobacterial florae in a host is modified and the behavior of a physiological molecule in the liver is controlled properly. A thermophilic bacterium probiotic which can also act in the case where the properties of animal species or an aging phenomenon in the intestine is observed is used. By using this thermophilic bacterium probiotic, the population of enterobacterial florae in a host is modified and the behavior of enterobacterial florae is controlled properly. For these reasons, a bacterium having Accession No. NITE BP-863, which is one of thermophilic Bacillus bacteria, is used. Accession No. NITE BP-863 has been internationally deposited by the present inventors with National Institute of Technology and Evaluation (NITE) Patent Microorganisms Depositary (NPMD) (#122, 2-5-8 Kazusakamatari, Kisarazu-shi, Chiba, Japan) on Jan. 15, 2010 (Accession No. BP-863).

The present invention also includes a method for producing a preparation which can exert a function of a probiotic or prebiotic depending on diatheses even when the preparation is prepared using a mixture of microorganisms.

The invention described in claim 1 is a microbial preparation for controlling the proportion of the population of bacteria belonging to the division Bacteroidetes, Firmicutes or Proteobacteria or the proportion of the population of bacteria belonging to the genus Clostridium, Lactobacillus, Bifidobacterium or Bacteroidetes in enterobacterial florae in an animal body, and for controlling the concentration of a functional molecule contained in a living body, the microbial preparation containing a microorganism P01931 (Accession No. BP-1931; internationally deposited with National Institute of Technology and Evaluation (NITE) Patent Microorganisms Depositary (NPMD) (#122, 2-5-8 Kazusakamatari, Kisarazu-shi. Chiba. Japan) on Sep. 4, 2014), or MK-01A (Accession No. BP-02066; internationally deposited with National Institute of Technology and Evaluation (NITE—) Patent Microorganisms Depositary (NPMD) (#122, 2-5-8 Kazusakamatari, Kisarazu-shi, Chiba, Japan) on Jun. 17, 2015), or MK-03A (Accession No. BP-02067; internationally deposited with National Institute of Technology and Evaluation (NITE) Patent Microorganisms Depositary (NPMD) (#122, 2-5-8 Kazusakamatari, Kisarazu-shi, Chiba. Japan) on Jun. 17, 2015), or a component of the microorganism.

The invention described in claim 2 is a microbial preparation for reducing bacteria belonging to at least one genus selected from the genera Enterococcus, Streptococcus and Clostridium cluster XI that increases with age and fatness among opportunistic infection bacteria in enterobacterial florae confirmed in individual animal species, the microbial preparation containing a microorganism BP-863 or a component of the microorganism BP-863.

The invention described in claim 3 is a microbial preparation having a function recited in claim 1 or 2 and capable of increasing Lactobacillus amylovorans in chickens, pigs and other animals.

The invention described in claim 4 is a microbial preparation having a function recited in any one of claims 1 to 3 and capable of increasing the diversity of a bacterial flora.

The invention described in claim 5 is a microbial preparation having a function recited in any one of claims 1 to 4 and capable of controlling the function of enterobacterial florae and a physiological function in an animal body depending on a diathesis associated with susceptibility to fatness or insusceptibility to fatness.

The invention described in claim 6 is a health food capable of reducing the amount of an antibiotic to be used and exhibiting a diathesis-improving function depending on the diathesis of an animal body and a human body by utilizing a function recited in any one of claims 1 to 5.

The invention described in claim 7 is a medicine capable of reducing the amount of an antibiotic to be used and exhibiting a diathesis-improving function depending on the diathesis of an animal body and a human body by utilizing a function recited in any one of claims 1 to 5.

### Advantageous Effects of Invention

In the context of the present application, it becomes possible to use a tailor-made type probiotic or prebiotic on the basis of genetic backgrounds of host animals, and therefore the spread of the probiotic or prebiotic is considered to be greatly effective. That is, for human bodies, it is conceived to deal depending on the difference in human races, the difference in districts, the difference in eating habits or the difference in disease types. In animals, it is obvious that the enterobacterial florae and diatheses vary depending on whether the animals are pet animals, farm animals or domestic poultry. Therefore, it becomes possible to establish a probiotic or prebiotic depending on the desired purpose with the above-mentioned differences taken into consideration. In addition, according to the present invention, it also becomes possible to control the microbial structure of an enterobacterial flora at ambient temperature inherent to hosts and the concentration of a biological molecule involved in a physiological function, depending on genetic backgrounds of animals, i.e., the property of a fatness-susceptible or fatness-insusceptible diathesis. Furthermore, it becomes possible to develop and spread a probiotic which can act efficiently depending on the diatheses of animals. Furthermore, it also becomes possible to control the increase or decrease in diversity of an enterobacterial flora, particularly to reduce opportunistic infection bacteria, depending on the genetic background-related diatheses of animals or the microbial structure of the enterobacterial flora at ambient temperature inherent to the hosts, thereby maintaining the population of useful bacteria.

We can increase the diversity of an enterobacterial flora significantly and particularly can reduce opportunistic infection bacteria in chickens, pigs and dogs by using a feed containing the BP-863. For example, the conventional problems can be solved by reducing the population of bacteria belonging to the genus Enteroccocus in chickens, the population of bacteria belonging to the genus Streptococcus in pigs and the population of bacteria belonging to the genus Clostoridium, which normally increases in old dogs, in dogs and by administering a species-specific useful bacterium, which tends to be decreased in these animal species, in combination. In this regard, the diatheses of animals vary depending on genetic backgrounds of the animals, and the microbial structure of an enterobacterial flora inherent in a host and the behavior of the concentration of a biological molecule sometimes vary depending on the diathesis of the host. In this case, it is needed to administer a proper probiotic. Thus, studies on diathesis-dependent probiotics which can control the diathesis-dependent properties are future challenges. There is also a case where an antibiotic is used as a control for an enterobacterial flora. In this case, the diversity of the enterobacterial flora may be lost and a possibility of the occurrence of adverse effects in intestinal environments is concerned. In this case, the administration of a proper probiotic is needed.

### BRIEF DESCRIPTION OF THE DRAWINGS

FIG. 1 shows a conceptual diagram of a diathesis-dependent probiotic.

FIG. 2 shows thermophilic bacteria which can be used in a diathesis-dependent probiotic and a standard bacterium strain for the thermophilic bacteria.

FIG. 3 shows the regulation of the glycolytic system in a liver using a diathesis-dependent probiotic.

FIG. 4 shows the regulation of β-oxidation and the TCA cycle in a liver using a diathesis-dependent probiotic.

FIG. 5 shows the regulation of the urea cycle in a liver using a diathesis-dependent probiotic.

FIG. 6 shows a conceptual diagram illustrating the control of enterobacterial flora without relying on the use of an antibiotic.

FIG. 7 shows a weight gain rate in a piglet receiving oral feeding of BP-863.

FIG. 8 shows a diversity of a bacterial flora in feces from a piglet receiving oral feeding of BP-863.

FIG. 9 shows the behavior of an opportunistic infection bacterium in feces from a piglet.

FIG. 10 shows the behavior of an opportunistic infection bacterium and a diversity of a bacterial flora in feces from a chicken.

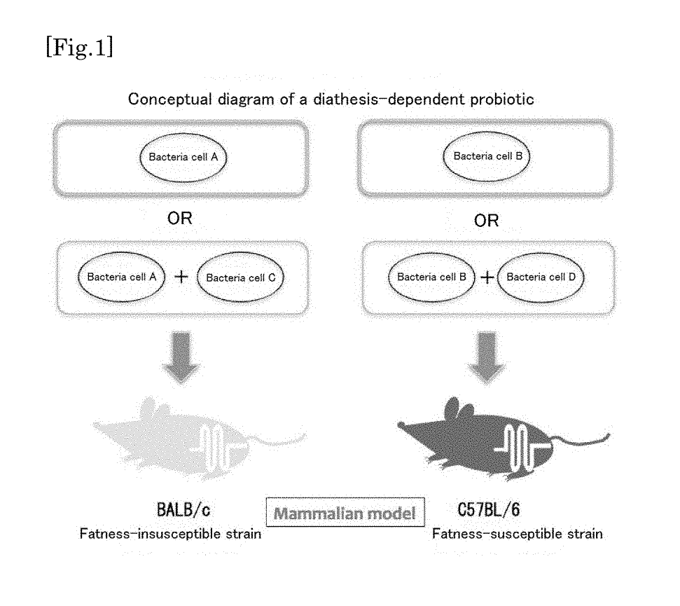

### DESCRIPTION OF EMBODIMENT

Next, the embodiment of the present invention will be described. However, the present invention is not limited to the embodiment.

Examples of the microorganism to be used in the present invention include thermophilic microorganisms of multiple organism species. Specific examples of the organism species include Bacillus coagulans and Bacillus thermoamylovorans, and related species thereof. The microorganism to be used in the present invention is particularly preferably the microorganism having Accession No. NITE P-01931 and/or a microorganism having Accession No. NITE BP-1051 (internationally deposited with National Institute of Technology and Evaluation (NITE) Patent Microorganisms Depositary (NPMD) (2-5-8 Kazusakamatari, Kisarazu-shi, Chiba, Japan) on Jan. 18, 2011) and/or the microorganism having Accession No. NITE BP-863 and/or a mixture of microorganisms MK-01 of which the accession was refused by National Institute of Technology and Evaluation (NITE) Patent Microorganisms Depositary (NPMD) because the strain was a mixture of microorganisms (Accession Refusal Notice No. 2014-0319) and/or a mixture of microorganisms MK-03 of which the accession was refused by National Institute of Technology and Evaluation (NITE) Patent Microorganisms Depositary (NPMD) because the strain was a mixture of microorganisms (Accession Refusal Notice No. 2014-0321). The species relating to Bacillus thermoamylovorans, particularly the group of microorganisms to be used in the present invention is preferably microorganisms having Accession No. NITE BP-863.

Examples of the microorganism that can be mixed with the microbial material to be used in the present invention include microorganisms belonging to the genera Lactobacillus and Bifidobacterium and thermophilic microorganisms belonging to the genera Bacillus, Lysinibacillus, Virgibacillus, Anoxybacillus and Paenibacillus. The desired physiological activity can also be achieved when Thermophiles inoculum MIROKU H2K including microorganisms belonging the genera Meiothermus. Vulcanithermus, Thermus and Oceanobacillus in the division Deinococcus-Thermus are co-present. The accession of this group of microorganisms Thermophiles inoculum MIROKU H2K was refused in National Institute of Technology and Evaluation (NITE) Patent Microorganisms Depositary (NPMD) because the strain was a mixture of microorganisms and could not be cultured easily, and therefore have been stored in Miroku Co., Ltd. (Kitsuki-shi, Ohita, Japan). As the group of microorganisms that can be co-present, microorganisms having Accession No. PTA-1773 which have been internationally deposited with ATCC (American Type Culture Collection, 10801 University Boulevard Manassas, Va. 20110-2209, USA) on May 1, 2000 can also be used. In addition, microorganisms that have been deposited with National Institute of Technology and Evaluation (NITE) Patent Microorganisms Depositary (NPMD) as a mixture of microorganisms under Accession No. NITE BP-1051 can also be used.

The Accession No. PTA-1773 is a thermophilic bacteria species.

In the preparation according to the present invention, a microorganism of each of the above-mentioned bacteria strains or a functional component derived from the microorganism is preferably contained in an amount of about 10 cells/g to about 10⁹ cells/g.

We have found that a thermophilic bacterium causes different physiological reactions in mice of different strains, using the above-mentioned microorganisms. When cells of each of the bacterium are administered to a mouse of a fatness-susceptible strain to a mouse of a fatness-insusceptible strain, the populations of bacteria belonging to the division Bacteroidetes, bacteria belonging to the genus Clostridium and bacteria belonging to the genus Lactobacillus in a microflora in the intestine can be controlled and the concentration of a functional molecule in a liver can also be controlled. In bacteria belonging to a single strain, there are a bacterial strain that tends to exert the effect thereof both on mice of a fatness-susceptible strain and mice of a fatness-insusceptible strain in the same manner and a bacterial strain that tends to exert the effect thereof on mice of a fatness-susceptible strain and mice of a fatness-insusceptible strain in a quite opposite manner. By utilizing these natures, the bacteria are used as diathesis-dependent probiotics and consequently the conventional problems can be solved.

The diversity of an enterobacterial flora can be increased significantly and particularly opportunistic infection bacteria can be reduced utilizing the above-mentioned microorganisms and by using a feed containing the BP-863 in chickens, pigs and dogs. For example, the conventional problems can be solved by reducing the population of bacteria belonging to the genus Enteroccocus in chickens, the population of bacteria belonging to the genus Streptococcus in pigs and the population of bacteria belonging to the genus Clostoridium, which normally increases in old dogs, in dogs. By utilizing these natures, the bacteria are used as probiotics capable of controlling an enterobacterial flora without relying on an antibiotic and consequently the conventional problems can be solved.

### Example 1

In a breeding test under a high-fat diet, the test was carried out using the formulations shown in Table 1.

TABLE 1

| Formulation | | High-fat diet (lard) | High-fat diet |
|---|---|---|---|
| General | Moisture | 6.2 g | 6.2 g |
| components | Protein | 18.9 g | 25.5 g |
| | Fat | 24.2 g | 32 g |
| | Ash | 4.9 g | 4 g |
| | Fiber | 2.3 g | 2.9 g |
| | Carbohydrate | 43.5 g | 29.4 g |

| | | |
|---|---|---|
| Total | 100 g | 100 g |

※ High-fat diet (lard) contained 20% of lard

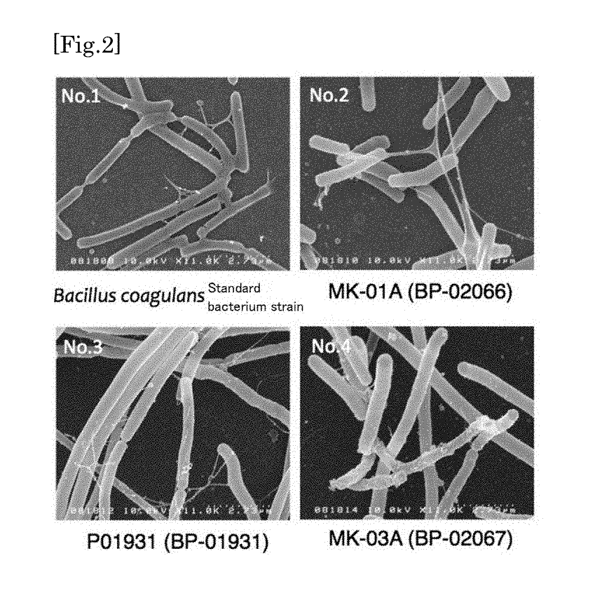

BALB/c and C57BL/6 mice (male, three-week old) were introduced and then raised preliminary for 5 days, and then an experiment started. The four groups (1) to (4) mentioned below were provided for each of the mouse strains, i.e., eight groups in total: (1) a group raised with a high-fat diet (lard) (a control group) (symbol mA for BALB/c) (symbol mE for C57BL/6); (2) a group raised with a high-fat diet (lard)+ with the addition of a thermophilic bacterium MK01 solution to drinking water (symbol mB for BALB/c) (symbol mF for C57BL/6); (3) a group raised with a high-fat diet (lard)+ with the addition of a thermophilic bacterium P01931 solution to drinking water (symbol mC for BALB/c) (symbol mG for C57BL/6); and (4) a group raised with a high-fat diet (lard)+ with the addition of a thermophilic bacterium MK03A solution to drinking water (symbol mD for BALB/c) (symbol mH for C57BL/6). A group of mice, which was composed of five mice, was raised in one cage. A formulated feed (MF manufactured by Oriental Yeast Co., Ltd.) was used as a standard feed, and the high-fat diet was produced by KBT Oriental Co., Ltd. (Tosu-shi, Saga, Japan), in which the fat content was adjusted to 24% (wherein lard made up 20%). The mice were allowed to take tap water ad libitum as the drinking water. In groups other than the control group, a 1.0% solution of the corresponding thermophilic bacterium was added to the drinking water. The mice were allowed to take a feed ad libitum within an intake limitation of 25 g per day. The mice were raised for 2 months, and were then subjected to the measurement of body weights, the collection of blood or the like, anatomy and the collection of livers and feces. The livers and feces were subjected to a metabolomic analysis and a bacterial flora analysis. The bacteria strains are shown in FIG. 2. Genetically, a 16SrDNA sequence in each of the bacteria strains was exactly the same as that in Bacillus coagulans (ATCC) that is a standard strain. However, the bacteria strains were different from each other morphologically.

As the results of the bacterial flora analysis, in the BALB/c mice, the change in bacterial flora in feces was confirmed in four groups, as shown in Tables 2 and 3. Especially in the mD group in which the body weight increasing tendency was low, a tendency that the populations of the bacteria belonging to the genera Clostridium and Lactobacillus were increased was confirmed.

TABLE 2
Bacterial flora data of division level

| | |
|---|---|
| BALB/c | Average |

| Phylum | mA Cf | mB Cf | mC Cf | mD Cf |
|---|---|---|---|---|
| Firmicutes | 1201.75 | 928.25 | 1433.5 | 1916.25 |
| Actinobacteria | 935.25 | 967.5 | 367 | 252.75 |
| Bacteroidetes | 367.5 | 723 | 652 | 438.5 |
| Proteobacteria | 14.5 | 25.25 | 40.75 | 40 |
| Deferribacteres | 1.25 | 5.5 | 12.5 | 4.25 |

TABLE 3
Bacterial flora data of genus level

| | |
|---|---|
| BALB/c | Average |

| Genus | mA Cf | mB Cf | mC Cf | mD Cf |
|---|---|---|---|---|
| Clostridium | 950.5 | 518 | 738.25 | 1248.25 |
| Bifidobacterium | 887.5 | 868.25 | 313 | 196.25 |
| Lactobacillus | 36.5 | 60.25 | 81.75 | 199.25 |
| Bacteroides | 28 | 86 | 83 | 40.25 |
| Robinsoniella | 8.25 | 18 | 16 | 18.25 |

As the results of the bacterial flora analysis, in the C57BL/6 mice, the change in bacterial flora in feces was confirmed in four groups, as shown in Tables 4 and 5. Especially in the mG group in which the body weight increasing tendency was low, a tendency that the populations of the bacteria belonging to the genera Clostridium and Lactobacillus were increased was confirmed.

TABLE 4

| | |
|---|---|
| C57BL/6 | Average |

| Phylum | mE Cf | mF Cf | mG Cf | mH Cf |
|---|---|---|---|---|
| Firmicutes | 1351.5 | 1273 | 1676 | 1365 |
| Bacteroidetes | 897.25 | 937 | 739.5 | 715.25 |
| Actinobacteria | 41.75 | 107.25 | 27 | 43 |
| Proteobacteria | 23.5 | 24 | 8.5 | 14.75 |
| Verrucomicrobia | 7.25 | 0.75 | 4 | 1.25 |

TABLE 5

| | |
|---|---|
| C57BL/6 | Average |

| Genus | mE Cf | mF Cf | mG Cf | mH Cf |
|---|---|---|---|---|
| Lactobacillus | 593 | 137.75 | 793 | 160.75 |
| Clostridium | 19.25 | 66.75 | 43.75 | 54.5 |
| Robinsoniella | 44.25 | 73.25 | 41.5 | 17 |
| Bacteroides | 45 | 47.25 | 27.25 | 29 |
| Bifidobacterium | 7.25 | 81 | 2.75 | 18.5 |

The analysis on diversity of the bacterial flora was confirmed by two kinds of methods. As a result, it was found that the diversity tended to increase in both of the bacteria strain administration groups, as shown in Tables 6 and 7, and therefore both of the groups were not different from each other with respect to this matter.

TABLE 6
Diversity analysis of bacterial flora

| | mA | mB | mC | mD |
|---|---|---|---|---|
| Index | (control) | (MK-01A) | (P01931) | (MK-03A) |
| OTU number | 100 | 111 | 132 | 124 |
| Chaol | 100 | 103 | 116 | 132 |

TABLE 7
Diversity analysis of bacterial flora

| | mE | mF | mG | mH |
|---|---|---|---|---|
| Index | (control) | (MK-01A) | (P01931) | (MK-03A) |
| OTU number | 100 | 121 | 106 | 116 |
| Chaol | 100 | 116 | 100 | 114 |

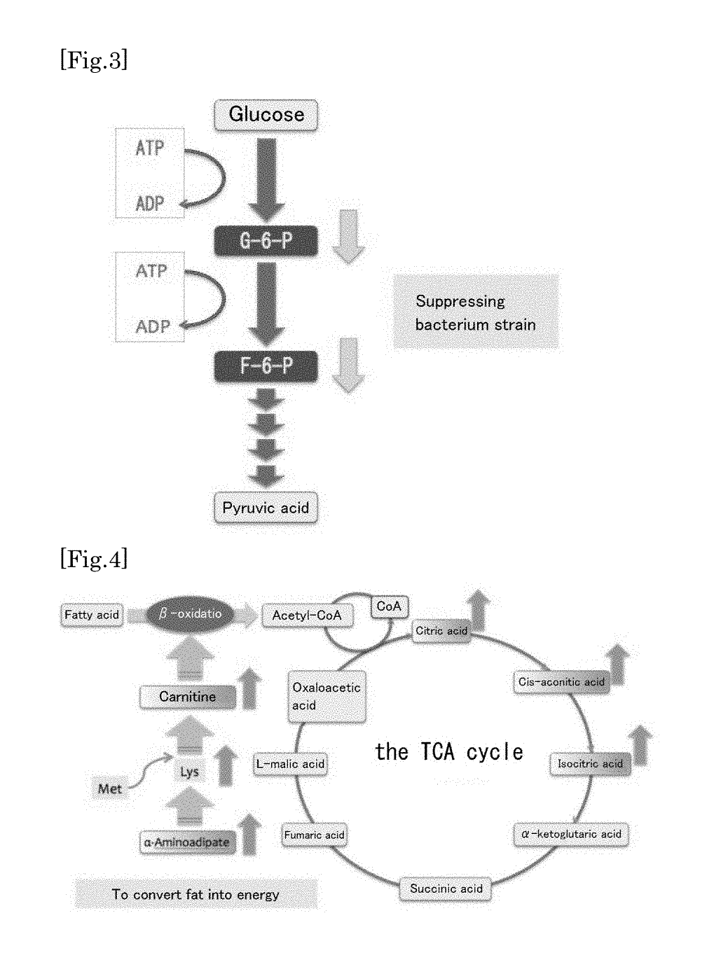

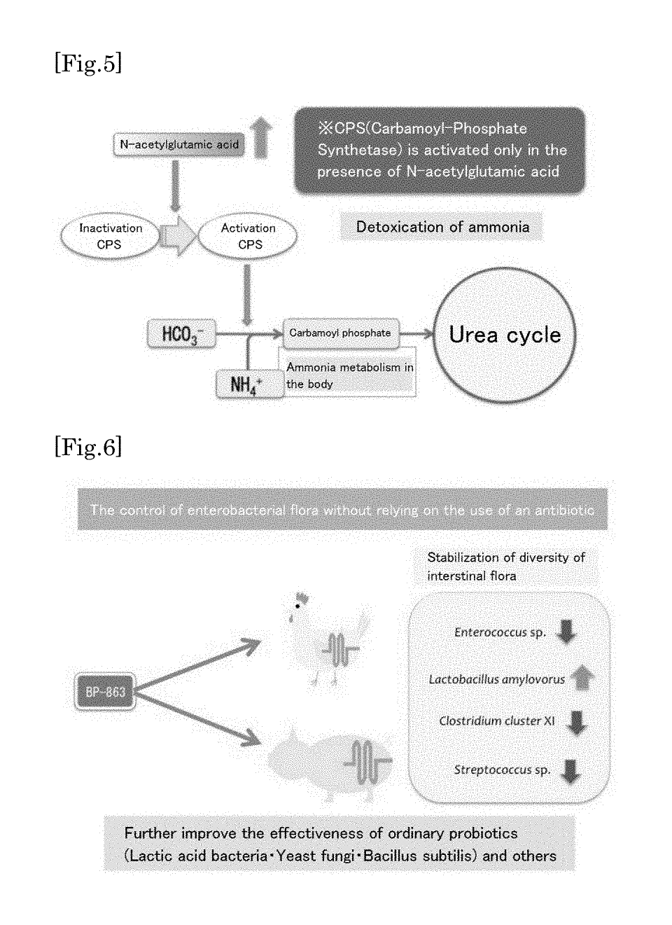

Next, the metabolomic analysis on a liver was carried out by CE-MS. As a result, the concentrations of biological functional molecules were different among the groups, as shown in Table 8. The results were not necessarily the same in some of the strains. In the analysis with respect to the glycolytic system, the decreasing tendency was confirmed in the production of F6P and G6P regardless of the types of the strains of the mice, as shown in FIG. 3. In contrast, in the analysis with respect to the TCA cycle, the increasing tendency was confirmed only in the mD group to which the MK03A strain was administered, but no change was observed in the other groups.

TABLE 8
Metabolomic analysis data on a liver

| Functional molecule | BALB/c 3 W | | | | C57BL/6 3 W | | | |
|---|---|---|---|---|---|---|---|---|
| in a liver | Control | MK01A | P01931 | MK03A | Control | MK01A | P01931 | MK03A |
| Allantoin | 100 | 98 | 76 | 90 | 100 | 101 | 104 | 100 |
| alpha-Aminoadipate | 100 | 71 | 78 | 74 | 100 | 98 | 82 | 99 |
| Betaine | 100 | 73 | 98 | 173 | 100 | 105 | 95 | 71 |
| Carnitine | 100 | 92 | 93 | 119 | 100 | 102 | 99 | 97 |
| cis-Aconitate | 100 | 98 | 87 | 138 | 100 | 95 | 102 | 87 |
| Choline | 100 | 107 | 120 | 151 | 100 | 90 | 103 | 120 |
| F6P | 100 | 103 | 73 | 27 | 100 | 85 | 86 | 42 |
| G6P | 100 | 110 | 70 | 30 | 100 | 106 | 91 | 39 |
| GABA | 100 | 92 | 111 | 176 | 100 | 174 | 306 | 385 |
| Gluconate | 100 | 125 | 120 | 142 | 100 | 92 | 89 | 106 |
| Hypoxanthine | 100 | 123 | 137 | 196 | 100 | 125 | 166 | 177 |
| Inosine | 100 | 112 | 116 | 115 | 100 | 126 | 125 | 105 |
| Isethionate | 100 | 113 | 119 | 168 | 100 | 98 | 108 | 105 |
| Lys | 100 | 101 | 113 | 141 | 100 | 119 | 151 | 186 |
| Met | 100 | 95 | 102 | 204 | 100 | 118 | 151 | 175 |
| N-Acetylglucosamine | 100 | 110 | 135 | 231 | 100 | 147 | 232 | 262 |
| N-Acetylglutamate | 100 | 77 | 61 | 81 | 100 | 90 | 69 | 73 |
| Pantothenate | 100 | 121 | 129 | 203 | 100 | 142 | 187 | 254 |
| Thiamine | 100 | 136 | 145 | 176 | 100 | 111 | 119 | 107 |
| Xanthine | 100 | 109 | 122 | 163 | 100 | 117 | 135 | 161 |

BALB/c and C57BL/6 mice (male, three-week old; and male, eight-week old) were introduced and then raised preliminary for 5 days, and then an experiment started. The three groups (1) to (3) mentioned below were provided: (1) a group raised in a normal manner (a control group); (2) a group with the addition of the BP-863; and (3) a group with the addition of a mixed solution to drinking water. A group of mice, which was composed of five mice, was raised in one cage. The mice were allowed to take tap water ad libitum as the drinking water, and were also allowed to take a feed ad libitum. The mice were raised for 3 months, and were then subjected to the measurement of body weights, the collection of blood or the like, anatomy and the collection of livers. The liver was subjected to a metabolomic analysis.

As the result of the blood analysis, the concentrations of leptin in serum in the C57BL′6 mice were higher than those in the BALB/c mice, but the C57BL/6 mice were likely to gain much body weights compared with the BALB/c mice.

As the result of the metabolomic analysis on livers, the same tendency was not observed depending on the age in weeks of the mice at the time of starting of administration and the strain of the mice, as shown in Tables 9 and 10.

TABLE 9

| Functional molecule | B6 3 w | | | BALB/c 3 w | | |
|---|---|---|---|---|---|---|
| in a liver | High-fat diet | BP-863 | Mixed slution | High-fat diet | BP-863 | Mixed slution |
| Allantoin | 100 | 96 | 109 | 100 | 123 | 101 |
| alpha-Aminoadipate | 100 | 289 | 144 | 100 | 178 | 147 |
| Betaine | 100 | 91 | 142 | 100 | 146 | 161 |
| Carnitine | 100 | 130 | 118 | 100 | 110 | 103 |
| Choline | 100 | 86 | 95 | 100 | 118 | 116 |
| cis-Aconitate | 100 | 217 | 377 | 100 | 469 | 498 |
| F6P | 100 | 101 | 61 | 100 | 88 | 75 |
| G6P | 100 | 94 | 57 | 100 | 100 | 76 |
| GABA | 100 | 76 | 104 | 100 | 67 | 64 |
| Gluconate | 100 | 88 | 92 | 100 | 112 | 132 |
| Hypoxanthine | 100 | 81 | 94 | 100 | 82 | 151 |
| Inosine | 100 | 91 | 93 | 100 | 108 | 95 |
| Isethionate | 100 | 106 | 82 | 100 | 133 | 114 |
| Lys | 100 | 91 | 98 | 100 | 105 | 138 |
| Met | 100 | 86 | 121 | 100 | 89 | 112 |
| N-Acetylglucosamine | 100 | 84 | 89 | 100 | 78 | 87 |
| N-Acetylglutamate | 100 | 171 | 139 | 100 | 190 | 170 |
| Pantothenate | 100 | 72 | 71 | 100 | 110 | 88 |
| Thiamine | 100 | 82 | 92 | 100 | 119 | 111 |
| Xanthosine | 100 | 93 | 102 | 100 | 109 | 104 |

TABLE 10

| Functional molecule | B6 8 w | | | BALB/c 8 w | | |
|---|---|---|---|---|---|---|
| in a liver | High-fat diet | BP-863 | Mixed slution | High-fat diet | BP-863 | Mixed slution |
| Allantoin | 100 | 94 | 98 | 100 | 98 | 87 |
| alpha-Aminoadipate | 100 | 104 | 139 | 100 | 153 | 152 |
| Betaine | 100 | 70 | 53 | 100 | 144 | 128 |
| Carnitine | 100 | 99 | 112 | 100 | 97 | 97 |
| Choline | 100 | 106 | 120 | 100 | 161 | 120 |
| cis-Aconitate | 100 | 98 | 120 | 100 | 447 | 1076 |
| F6P | 100 | 94 | 79 | 100 | 62 | 51 |
| G6P | 100 | 98 | 86 | 100 | 71 | 59 |
| GABA | 100 | 133 | 158 | 100 | 78 | 90 |
| Gluconate | 100 | 48 | 108 | 100 | 145 | 111 |
| Hypoxanthine | 100 | 111 | 139 | 100 | 97 | 102 |
| Inosine | 100 | 101 | 116 | 100 | 99 | 109 |
| Isethionate | 100 | 230 | 252 | 100 | 80 | 89 |
| Lys | 100 | 91 | 126 | 100 | 134 | 150 |
| Met | 100 | 95 | 118 | 100 | 119 | 111 |
| N-Acetylglucosamine | 100 | 91 | 136 | 100 | 98 | 121 |
| N-Acetylglutamate | 100 | 97 | 158 | 100 | 152 | 122 |
| Pantothenate | 100 | 29 | 19 | 100 | 120 | 104 |
| Thiamine | 100 | 116 | 143 | 100 | 99 | 87 |
| Xanthosine | 100 | 111 | 133 | 100 | 116 | 137 |

The above results indicate that almost similar physiological reactions were induced by the bacterial species under a high-fat diet condition both in the mice each having a fatness-susceptible diathesis (C57BL/6) and the mice each having a fatness-insusceptible diathesis (BALB/c). As shown in Table 1, it was found that almost similar physiological reactions, including the increase in bacteria belonging to the division Bacteroidetes in the microflora in the intestine and the suppression of the glycolytic system, the activation of the TCA cycle and the activation of the urea cycle in the liver, were induced. The increase in free amino acids was confirmed only in the mice of a fatness-insusceptible strain. The reaction was observed significantly in newborn three-week-old mice, while a slightly different reaction was observed in the eight-week old mice.

Therefore, it was concluded that the technique of the present invention is novel over the prior art.

### Example 2

A feed was prepared by blending the BP-863 to a feed in an amount of 10² per 1 kg of the feed, and the resultant feed was fed for 3 weeks to piglets which were born from the same mother pig and were about 30 day old from the time of birth.

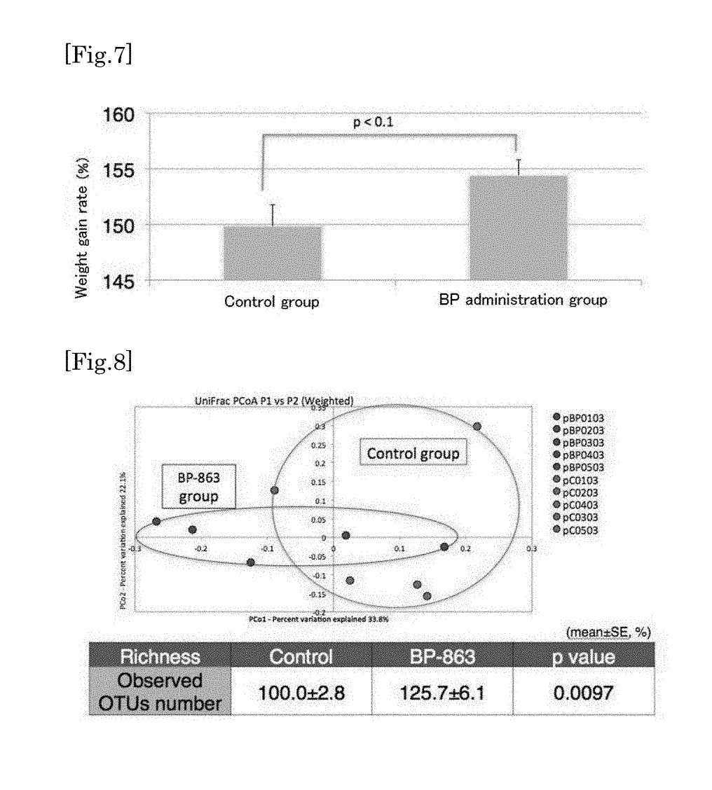

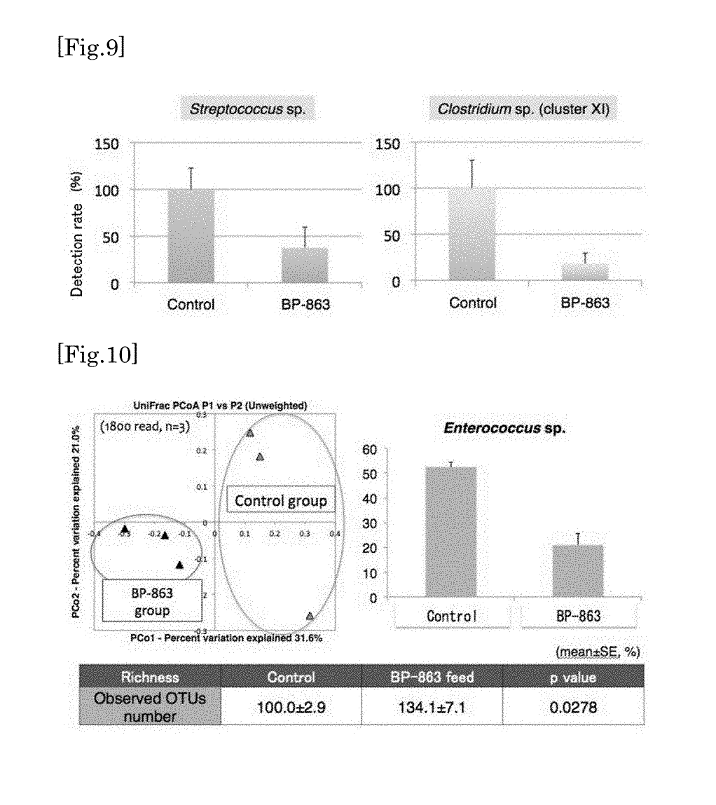

As a result, it was confirmed that the weight gain rate in the piglets tended to increase by about 5% (see FIG. 7). At this point of time, total DNA was extracted from porcine feces, and then subjected to the comprehensive analysis on bacterial 16SrDNA using a next-generation sequencer. That is, the analysis of a bacterial flora in porcine feces was carried out with respect to bacterial 16SrDNA using a next-generation sequencer through a 3000 read analysis. As a result, a change in bacterial flora was confirmed between pigs to which a feed containing the BP-863 was fed and pigs in a non-administered group, and the population of opportunistic infection bacteria was remarkably reduced. On the other hand, the number of detected bacterial species was significantly increased in the BP-863-fed zone in the 3000 reads. This analysis was carried out employing the method described in DNA Res. June; 20(3): 241-253, 2013 and the method described in DNA Res. February; 21(1): 15-25, 2014.

Specifically, as the result of the UniFrac analysis, the bacterial flora of the BP-863-unadministered group and the bacterial flora of the BP-863-administered group were significantly different from each other, as shown in FIG. 8. In addition, the number of detected units having different sequences (OUT) was increased in the BP-863-administered group, which demonstrates that diversity was increased. As the result of the detailed analysis, the population of bacterial species related to bacteria belonging to the genus Streptococcus which is assumed to be an opportunistic infection bacterium was remarkably reduced. It was also confirmed that the population of bacterial species belonging to cluster XI, among the genus Clostoridium, tended to be reduced. An example of the bacterium belonging to the genus Clostoridium, cluster XI is Clostridium mayombei. Clostoridium cluster XI is known as one of bacteria that have been assumed to be increased during the experience of fatness in an experiment using mice (Nature July 4; 499(7456): 97-101, 2013).

As the bacteria that belong to the genus Streptococcus and were also expected to be reduced in pigs in other experiment systems, Streptococcus alactoriticus, Streptococcus galactotiticus, Streptococcus orisuis and Streptococcus hyointestinalis were conceived.

In the experiment, Lactobacillus amylovorus and a bacterium belonging to the genus Bifidobacterium, among lactic acid bacteria, tended to be increased.

In general, the increase in the population of bacterial species belonging to the genus Clostridium in the intestine is observed with age. The tendency of reduction in Clostridium XI in the enterobacterial florae in an animal body receiving the administration of BP-863 was also confirmed in old dogs.

### Example 3

Each of a feed containing the BP-863 and a feed not containing the BP-863 was fed to big chicks of egg-laying chickens for 18 weeks, and the bacterial flora in feces from each of the chicks was analyzed in the same manner as described in paragraph [0050].

The analysis of a bacterial flora in chicken feces was carried out with respect to bacterial 16SrDNA using a next-generation sequencer through a 1800 read analysis. As a result, a change in bacterial flora was confirmed between chickens to which a feed containing the BP-863 was fed and chickens of a non-administered group, and bacteria belonging to the genus Enterococuus, which are known as vancomycin-resistant bacteria, were remarkably reduced. On the other hand, the number of detected bacterial species was significantly increased in the BP-863-fed zone among the 1800 reads. Specifically, a significant difference in bacterial flora was confirmed between the BP-863-administered group and the BP-863-unadministered group, as shown in FIG. 10. In addition, the number of units having different sequences (OUT) was increased in the BP-863-administered group, which demonstrates that diversity was increased. Particularly, the population of Enterococcus gallinarum, which is one of bacteria belonging to the genus Enterococcus, was remarkably reduced. In this experiment, the tendency of increase in the population of Lactobacillus amylovorus was confirmed.

From these results, it was confirmed that the BP-863 has a tendency to control a bacterial flora and particularly increase the diversity of the bacterial flora, while it was demonstrated that the BP-863 increases the population of specific useful bacteria and promotes the decrease in population of specific opportunistic infection bacteria. When an antibiotic is administered, this tendency causes the death of many enterobacterial florae and therefore causes the loss of diversity of a bacterial flora. Therefore, it can be considered that the technique of the present invention is novel over the prior art. As mentioned above, in recent years, it has been found that the diversity of an enterobacterial flora is essential for the control of obesity or the prevention of various diseases. Therefore, it is considered that the effectiveness of the present invention is high. According to the present invention, it is expected that it becomes possible to reduce specific opportunistic infection bacteria without relying on the use of antibiotics, and it also becomes possible to produce the diversity of an enterobacterial flora regardless of the types of species of animals to prevent various diseases.

### Accession Numbers

### NITE BP-01931

### NITE BP-02066

### NITE BP-2067

### NITE BP-863

### NITE BP-1051

### ATCC PTA-1773

### Accession Refusal Notice No. 2014-0319

### Accession Refusal Notice No. 2014-0321

Thermophiles inoculum MIROKU M2K strain

1. A microbial preparation for controlling the proportion of the population of bacteria belonging to the division Bacteroidetes, Firmicutes or Proteobacteria or the proportion of the population of bacteria belonging to the genus Clostridium, Lactobacillus, Bifidobacterium or Bacteroidetes in enterobacterial florae in an animal body, and for controlling the concentration of a functional molecule contained in a living body, the microbial preparation containing a microorganism P01931 (International Accession No. BP-1931), MK-01A (International Accession No. BP-02066) or MK-03A (International Accession No. BP-02067) or a component of the microorganism.

2. A microbial preparation for reducing bacteria belonging to at least one genus selected from the genera Enterococcus, Streptococcus and Clostridium cluster XI that increases with age and fatness among opportunistic infection bacteria in enterobacterial florae confirmed in individual animal species, the microbial preparation containing a microorganism BP-863 or a component of the microorganism BP-863.

3. A microbial preparation having a function recited in claim 1 and capable of increasing Lactobacillus amylovorans in chickens, pigs and other animals.

4. A microbial preparation having a function recited in claim 1, and capable of increasing the diversity of a bacterial flora.

5. A microbial preparation having a function recited in claim 1, and capable of controlling the function of enterobacterial florae and a physiological function in an animal body depending on a diathesis associated with susceptibility to fatness or insusceptibility to fatness.

6. A health food capable of reducing the amount of an antibiotic to be used and exhibiting a diathesis-improving function depending on the diathesis of an animal body and a human body by utilizing a function recited in claim 1.

7. A medicine capable of reducing the amount of an antibiotic to be used and exhibiting a diathesis-improving function depending on the diathesis of an animal body and a human body by utilizing a function recited in claim 1.

8. A microbial preparation having a function recited in claim 2 and capable of increasing Lactobacillus amylovorans in chickens, pigs and other animals.

9. A microbial preparation having a function recited in claim 2 and capable of increasing the diversity of a bacterial flora.

10. A microbial preparation having a function recited in claim 3 and capable of increasing the diversity of a bacterial flora.

11. A microbial preparation having a function recited in claim 2 and capable of controlling the function of enterobacterial florae and a physiological function in an animal body depending on a diathesis associated with susceptibility to fatness or insusceptibility to fatness.

12. A microbial preparation having a function recited in claim 3 and capable of controlling the function of enterobacterial florae and a physiological function in an animal body depending on a diathesis associated with susceptibility to fatness or insusceptibility to fatness.

13. A microbial preparation having a function recited in claim 4 and capable of controlling the function of enterobacterial florae and a physiological function in an animal body depending on a diathesis associated with susceptibility to fatness or insusceptibility to fatness.

14. A health food capable of reducing the amount of an antibiotic to be used and exhibiting a diathesis-improving function depending on the diathesis of an animal body and a human body by utilizing a function recited in claim 2.

15. A health food capable of reducing the amount of an antibiotic to be used and exhibiting a diathesis-improving function depending on the diathesis of an animal body and a human body by utilizing a function recited in claim 3.

16. A health food capable of reducing the amount of an antibiotic to be used and exhibiting a diathesis-improving function depending on the diathesis of an animal body and a human body by utilizing a function recited in claim 4.

17. A health food capable of reducing the amount of an antibiotic to be used and exhibiting a diathesis-improving function depending on the diathesis of an animal body and a human body by utilizing a function recited in claim 5.

18. A medicine capable of reducing the amount of an antibiotic to be used and exhibiting a diathesis-improving function depending on the diathesis of an animal body and a human body by utilizing a function recited in claim 2.

19. A medicine capable of reducing the amount of an antibiotic to be used and exhibiting a diathesis-improving function depending on the diathesis of an animal body and a human body by utilizing a function recited in claim 3.

20. A medicine capable of reducing the amount of an antibiotic to be used and exhibiting a diathesis-improving function depending on the diathesis of an animal body and a human body by utilizing a function recited in claim 4.

21. A medicine capable of reducing the amount of an antibiotic to be used and exhibiting a diathesis-improving function depending on the diathesis of an animal body and a human body by utilizing a function recited in claim 5.
